(19) United States
(12) Patent Application Publication
(10) Pub. No.: US 2024/0050659 A1
(43) Pub. Date: Feb. 15, 2024

(54) HOUSING COMPONENT OF AN INJECTION DEVICE COMPRISING A MAGNIFIER

(71) Applicant: Sanofi, PARIS (FR)

(72) Inventors: Michael Helmer, Frankfurt am Main (DE); Martin Vitt, Frankfurt am Main (DE)

(21) Appl. No.: 18/267,522

(22) PCT Filed: Jan. 3, 2022
(86) PCT No.: PCT/EP2022/050002

§ 371 Date: Jun. 15, 2023

(30) Foreign Application Priority Data
Jan. 4, 2021 (EP) 21315003.0

(51) Int. Cl. A61M 5/24; A61M 5/20; A61M 5/31

(52) U.S. Cl. CPC A61M 5/24; A61M 5/20; A61M 5/31; A61M 2005/2006; A61M 2005/2403; A61M 2005/3125; A61M 2205/585

(57) ABSTRACT

An elongated housing component of an injection device, the elongated housing component including a body comprising a first longitudinal end, an intermediate section, and a second longitudinal end opposite to the first longitudinal end. The intermediate section is located longitudinally between the first longitudinal end and the second longitudinal end. The body includes a sidewall extending from the first longitudinal end to the second longitudinal end. The sidewall confining a hollow interior space sized to accommodate a medicament container. The elongated housing component including a magnifier extending through the sidewall or integrated into the sidewall to increase a visual appearance of at least a portion of the medicament container when arranged inside the hollow interior space.

### CROSS-REFERENCE TO RELATED APPLICATIONS

The present application is the national stage entry of International Patent Application No. PCT/EP2022/050002, filed on Jan. 3, 2022, and claims priority to Application No. EP 21315003.0, filed on Jan. 4, 2021, the disclosures of which are incorporated herein by reference.

### TECHNICAL FIELD

The present disclosure relates to the field of injection devices, in particular to an elongated housing component of injection devices, such as a handheld pen injectors and an injection device comprising a housing component equipped with a magnifier.

### BACKGROUND

Drug delivery devices for setting and dispensing a single or multiple doses of a liquid medicament are well-known. Generally, such devices have substantially a similar purpose as that of an ordinary syringe.

Drug delivery devices, such as pen-type injectors have to meet a number of user-specific requirements. For instance, with patients suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

Typically, such devices comprise a housing or a particular cartridge holder adapted to receive a medicament container, e.g. in form of a cartridge, at least partially filled with the medicament to be dispensed. Such devices further comprise a drive mechanism, usually having a displaceable piston rod to operably engage with a bung or piston of the cartridge. Typically, the drive mechanism comprises a piston rod being operable to apply a dispensing pressure onto the bung or piston of the cartridge, thereby displacing the piston or bung in a distal direction or dispensing direction to expel a predefined amount of the medicament, i.e. a dose, via a piercing assembly, e.g. comprising an injection needle. The injection needle, which is e.g. part of a needle hub releasably connectable to a distal end of the housing of the drug delivery device, is typically in fluid communication with the interior of the cartridge.

The medicament to be dispensed by the drug delivery device may be provided and contained in a multi-dose cartridge. Such cartridges typically comprise a vitreous barrel sealed in distal direction by means of a pierceable seal and being further sealed in proximal direction by the bung or piston. With reusable drug delivery devices an empty cartridge is replaceable by a new one. In contrast to that, drug delivery devices of disposable type are to be entirely discarded when the medicament in the cartridge has been dispensed or used-up.

An elongated housing component shaped and sized to accommodate a medicament container, such as a cartridge may comprise a transparent or translucent section that allows visual inspection of the content of the medicament container from outside the housing. Medicament containers are typically provided with a label or the like visual information, e.g. being indicative of medicament related information, such as the name, the type, the concentration and/or the best before date of the medicament. Especially with medicament containers comprising a comparatively small filling volume, such as 10 ml, 5 ml, 3 ml, 1.5 ml or even less, the outer surface of the medicament container only provides limited space for visually displaying respective information. Accordingly, the size of the visual information may be therefore quite small and difficult to read.

### SUMMARY

Certain embodiments of the present disclosure improve legibility of information provided on a medicament container. Legibility is improved at least when the medicament container is located inside a housing of the respective injection device. The improvements with regard to legibility come along with only a minimum of modifications of existing injection devices.

According to one aspect, the present disclosure relates to an elongated housing component of an injection device. The elongated housing component comprises a body. The body comprises a first longitudinal end, an intermediate section and a second longitudinal end. Hence, the body is an elongated body, wherein the first longitudinal end is a distal end and wherein the second longitudinal end is a proximal end. The second longitudinal end is located opposite to the first longitudinal end. The intermediate section is located longitudinally between the first longitudinal end and the second longitudinal end.

The body comprises a sidewall extending from the first longitudinal end to the second longitudinal end. The sidewall confines a hollow interior space of the body. The hollow interior space is sized to accommodate a medicament container, such as a tubular-shaped barrel, e.g. filled with a liquid medicament.

The elongated housing component further comprises a magnifier extending through the sidewall or being integrated into the sidewall. The magnifier is operable to increase a visual appearance of at least a portion of the medicament container when arranged inside the hollow interior space of the body. The magnifier is configured and shaped to increase the visual appearance of a portion of the medicament container when arranged inside the body and when viewed from outside the elongated housing component. Insofar, the magnifier is transparent for electromagnetic radiation in the visible spectral range. The sidewall may be also transparent.

The magnifier and the sidewall may be made from the same material. With some examples the magnifier and the sidewall are unitarily shaped. The magnifier and the sidewall may be integrally formed. Insofar, the magnifier may be integrated into the sidewall and hence integrated into the body of the elongated housing component. The magnifier may be immovably fixed to the body or integrated into the body. The magnifier may form or constitute a portion of the sidewall of the body of the elongated housing component. With other examples the magnifier and the sidewall are made from the same or different materials and may form separate parts or pieces to be assembled together. Here, the magnifier may comprise or may constitute an insert shaped and configured for insertion into a complementary-shaped recess or through opening of the sidewall. The magnifier may be exchangeably or interchangeably arranged inside, on or to the sidewall.

With some examples the elongated housing component is made of an injection moldable plastic material. Typically, the plastic material is a transparent plastic material. An injection moldable plastic material is beneficial to easily integrate the magnifier in the sidewall of the elongated housing component. The magnifier and the sidewall may be integrally formed.

With some examples the magnifier protrudes radially from the sidewall. It may protrude radially outwardly from the sidewall. With other examples an outside surface of the magnifier and an outside surface of the sidewall mutually align or flush, as seen in longitudinal, tangential or circumferential direction of the elongated body.

Providing of the magnifier in the region of the sidewall enables to improve legibility of e.g. a label provided on an outside surface or inside surface of the medicament container when arranged inside the elongated housing component. Typically, the label may be provided with information related to the medicament located inside the medicament container. Hence, the label may comprise a printed information, such as numbers, symbols or text.

With other examples the outside surface or inside surface of the medicament container may be provided with a scale, e.g. extending along the longitudinal direction when the medicament container is arranged inside the elongated housing component. The scale may be provided with numbers or symbols by way of which a filling level of the liquid medicament contained inside the medicament container can be visually inspected. In either way, either a label and/or a scale provided on or in the medicament container becomes easily discernible and/or visible from outside when viewed through the magnifier of the elongated housing component. In this way, patient safety for end-users making use of the injection device can be increased. Legibility of information provided on the medicament container can be improved.

Typically, the magnifier is an optical magnifier. The magnifier may be shaped and configured to provide an optical magnifying effect, such as a magnifying lens. Insofar, the magnifier may be of convex shape and/or may comprise a convex cross section.

With some examples the elongated housing component is a cartridge holder of a pen-type injection device. The injection device may be implemented as a handheld injection device. The elongated housing component is connectable, e.g. non-releasably or releasably connectable to a proximal housing component of the injection device, e.g. denoted as body of the injection device. Typically, the second longitudinal end of the elongated housing component is connectable to a further housing component of the injection device, such as a proximal housing component, while the elongated housing component forms or constitutes a distal housing component. With some examples, the first longitudinal end of the body of the elongated housing component does not only form or constitute a distal end of the elongated housing component but also forms or constitutes a distal end of the injection device.

Hence, the elongated housing component may be implemented as a distal housing component of the injection device. It may comprise a proximal connecting end provided at a proximal longitudinal end of the body. The further housing component of the injection device may be implemented as a proximal housing component. It may comprise a distal connecting end provided at a distal longitudinal end. Typically, the elongated housing component is connectable with its proximal connecting end to the distal connecting end of the further housing component. Insofar, and when mutually assembled the first housing component may form or constitute a longitudinal prolongation or extension protruding distally from the further housing component.

The first longitudinal end of the body is configured and shaped to engage with an injection needle. Typically, the injection needle comprises a needle hub for releasably connecting to the first longitudinal end of the body of the elongated housing component. Typically, the first longitudinal end of the body comprises a through opening in a distal end face configured and shaped to receive a proximally extending tipped end of the injection needle of the needle assembly. The medicament container located inside the body may comprise a pierceable seal at a distal end that is to be pierced and/or penetrated by the proximal end of the double-tipped needle of the needle assembly when the needle assembly is attached to the first longitudinal end of the body.

According to a further example the magnifier comprises a bulged section extending outwardly from the sidewall. An outwardly extending bulged section of the magnifier provides an increased degree of visual or optical magnification of an item, e.g. of the medicament container located inside the hollow interior space.

According to a further example the magnifier is of elongated shape and extends parallel to a longitudinal axis of the body. The magnifier may comprise a constant and hence non-varying cross-section as seen in longitudinal direction. The elongated shape of the magnifier provides a visual magnification of an elongated section on the medicament container when arranged inside the hollow interior space. This is of particular benefit when for instance a medicament name or some other type of information is printed along the longitudinal direction, e.g. onto a longitudinally shaped barrel or label of the medicament container. In this way, at least an elongated stripe extending along the medicament container can be visually magnified rather homogeneously by the elongated magnifier when the medicament container is arranged inside the hollow interior space of the body of the elongated housing component.

According to a further example the magnifier is located in the intermediate section of the body. The magnifier may be located at a predefined longitudinal position between the first longitudinal end and the second longitudinal end. With other examples the magnifier can be located at one of the first longitudinal end and the second longitudinal end. With further examples there may be provided at least a first magnifier and a second magnifier located at a distance from each other on the sidewall of the body of the elongated housing component. The first and the second magnifier may be located at a longitudinal offset and/or at a circumferential offset from each other. In this way, different sections of the medicament container can be visually magnified when the medicament container is arranged inside the hollow interior space.

According to another example the magnifier extends along the intermediate section of the body. The magnifier may extend all along the intermediate section. The magnifier may extend non-interrupted between the first longitudinal end and the second longitudinal end of the body. Typically, the magnifier extends along a cylindrically shaped intermediate section of the body, which is configured and shaped to accommodate a cylindrical or tubular portion of the barrel of the medicament container configured for insertion into the hollow interior space. With such an elongated longitudinally extending magnifier a respective elongated portion or section of the medicament container can be visually inspected from outside the elongated housing component in a magnified way.

At or near the first longitudinal end and/or the second longitudinal end the body may comprise a diameter or cross section that differs from the diameter or cross section of the intermediate section. Typically, the intermediate section is a portion of the body of constant diameter as seen in longitudinal direction. Towards the first longitudinal end the intermediate section may adjoin a radially inwardly extending shoulder portion of the body. Towards the second proximal end the intermediate section may adjoin a proximal connector or connecting portion of the body, which is shaped and configured to mechanically engage with a correspondingly shaped connector of a further housing component of the injection device.

According to a further example the intermediate section comprises a first outer diameter and wherein the second longitudinal end comprises a second outer diameter. The first outer diameter is smaller than the second outer diameter. With some examples the first outer diameter may be larger than a third outer diameter. The third outer diameter may be provided at the first longitudinal end of the body.

In other words, a radial expansion, radial extent or radial dimension of the intermediate section is somewhat smaller compared to the radial extent or radial expansion of the second longitudinal end of the body. With some examples, the intermediate section of the body may be radially stepped down compared to the second longitudinal end.

With some examples the second longitudinal end is a proximal end of the body configured to engage or to connect with a distal end of a further housing component, e.g. a proximal housing component of the injection device. It should be noted here, that any reference to a radial diameter equally applies to a cross-section in the radial transverse plane.

Reducing the diameter or the transverse cross-section of the intermediate section compared to the second longitudinal end of the body provides a free space between an outside surface of the intermediate section of the sidewall and an inside surface of a protective cap of the injection device configured to cover at least a portion of the elongated housing component, particularly when the device is not in use. In this way and by reducing the radial extent or radial cross-section of the intermediate section compared to an inside diameter of the protective cap there is provided sufficient space for the magnifier to extend radially outwardly from the sidewall of the intermediate section, e.g. in form of a bulged section.

By reducing the diameter or the radial cross-section of the intermediate section at least in a region, where the magnifier is located, enables and provides the benefit to cover the respective portion of the elongated housing component with a protective cap, such as a pen cap. Typically, the radial size reduction of the intermediate section or of any other section of the sidewall of the body that is provided with the at least one magnifier is larger than or equal to a radial extent of the bulged section of the magnifier extending outwardly from the respective sidewall or sidewall section of the body of the elongated housing component.

In this way, an existing exterior design of the injection device, e.g. of a protective cap, can be maintained and an existing protective cap configured to cover and/or to receive the elongated housing component can be also used with the presently disclosed elongated housing component featuring the magnifier.

According to a further example the intermediate section comprises a polygonal cross-section with at least a first sidewall section, a second sidewall section and a third sidewall section mutually adjoining along a tangential direction. The first, the second and the third sidewall sections may each comprise a somewhat planar-shaped sidewall section. With other examples the first, the second and/or the third sidewall sections may each comprise a slightly bended or arched structure as seen in circumferential or tangential direction of the elongated housing component.

The first, the second and the third side wall sections may conform a somewhat triangular cross-section of the elongated housing component at least as seen from the exterior of the body. Insofar, the thickness of each one of the first, the second and the third side wall sections may vary as seen in tangential direction. Typically, and as seen in longitudinal direction the first, the second and the third sidewall sections comprise a non-varying thickness.

With the intermediate section comprising at least a first, a second and a third sidewall section the exterior circumference and in particular the exterior radial dimension of the intermediate section can be reduced compared to a tubular shaped intermediate section. This provides sufficient construction space for the magnifier extending radially outwardly from at least one of the first, the second and the third side wall section.

In principle, it is possible that each one of the first, the second and the third sidewall sections is provided with an own magnifier, e.g. extending all along the longitudinal extent of the first, the second and the third side wall sections of the intermediate section of the body. With some examples it may be sufficient when only one of the first, the second and the third sidewall sections is provided with the magnifier.

According to another example the first sidewall section adjoins in a first tangential direction to the second side wall section along or across a first ridge portion. The second sidewall section adjoins along the first tangential direction to the third side wall section along or across a second ridge portion. Furthermore, the first sidewall section adjoins in a second tangential direction, opposite to the first tangential direction, to the third side wall section along or across a third ridge portion. In other words, the third side wall section adjoins along the first tangential direction to the first sidewall section along the third ridge portion.

The sidewall sections are confined in tangential direction by respective ridge portions. The sidewall sections and the ridge portions may continuously and unalterably extend in longitudinal direction. In this way, the intermediate section with first, second and third sidewall sections as well as with first, second and third ridge portions comprises a continuous and constant cross-sectional profile as seen in longitudinal direction.

Typically, the ridge portion extending between adjacently located sidewall sections of the intermediate section comprises or constitutes a radial wall thickness that is larger than a radial wall thickness of the respective sidewall sections at a portion which is tangentially offset from the ridge portion. In this way, the first, the second and the third ridge portions provide a stiffening effect to the sidewall sections. The first, the second and/or the third sidewall sections comprise a minimum radial thickness in a region furthest away from the ridge portions, e.g. midway between circumferentially adjoining ridge portions.

In this way the hollow interior space confined in radial and tangential or circumferential direction by first, second and third sidewall portions may comprise a somewhat tubular shape with a tubular shaped inside surface. In this way a rather tubular shaped medicament container, e.g. a medicament container with a tubular-shaped barrel can be easily and smoothly inserted into the elongated housing component.

According to a further example the magnifier or the at least one magnifier is located on the first sidewall section between the first ridge portion and the third ridge portion. Typically, the magnifier is located tangentially midway between the first ridge portion and the third ridge portion on the first sidewall section. As described above the magnifier may be integrally formed with the first sidewall section. It may comprise a bulged section extending radially outwardly from the residual portion of the first sidewall section.

Providing of the magnifier at a distance from and between the first and the third ridge portions enables to provide a radially outwardly bulged section of the magnifier such that an outside surface of the bulged section of the magnifier is still located radially inside an imaginary tubular shaped shell that coincides with the radial positions of the first, the second and the third ridge portion.

In effect, the first, the second and the third ridge portions may be located on the corners of an imaginary triangle, wherein these corners are located on an imaginary circle, oval or rim that corresponds with an inside diameter of a protective cap of the injection device configured to cover the elongated housing component.

According to another example at least a first sidewall section comprises a convex shape as seen along the tangential or circumferential direction of the body. In this way, the sidewall section itself may already provide a visual or optical magnification at least to certain degree. The magnifier provided on or extending through the first sidewall section may thus increase the visual magnification. The convex shape of the first sidewall section may provide an attractive aesthetic design of the elongated housing component.

With some examples also the second sidewall section and/or the third sidewall section comprise a convex shape as seen along the tangential direction. Moreover, the respective ridge portions extending between the first, the second and the third sidewall sections may comprise a somewhat rounded shape as seen in tangential direction, thus providing an attractive exterior design of the elongated housing component.

According to a further example the body comprises at least one through opening extending through the sidewall to provide access to the interior space. The through opening is typically sized to receive at least a fingertip of a user. In this way, a user may at least slightly make contact with the medicament container when the latter is located inside the hollow interior space. Since the magnifier is typically provided at a well-defined or predefined tangential position on the body of the elongated housing component it may be necessary to rotate the e.g. tubular shaped medicament container inside the hollow interior space so as to provide a radial overlap between the magnifier of the body and a portion of the medicament container to be magnified.

With typical examples, the medicament container comprises an elongated, e.g. a tubular-shaped barrel provided with a label featuring visual information on a particular tangential portion of the medicament container. However, the medicament container may be circular symmetric with regard to its main axis or center axis. Thus and in order to bring the label or the respective information provided on the medicament container in an overlapping configuration with the magnifier of the elongated housing component it may be required to rotate the medicament container with regard to its longitudinal axis relative to the housing component.

Providing at least one through opening extending through the sidewall of the body provides respective access, e.g. for a fingertip of a user to induce a respective torque or momentum onto the outside surface of the medicament container, thereby inducing a respective rotation of the medicament container relative to the body of the elongated housing component.

According to a further example the body comprises a first through opening and a second through opening. The first through opening and the second through opening both both through the sidewall. Typically, the first through opening is located opposite, e.g. diametrically opposite to the second through opening. Providing a first and a second through opening at opposite sections of the sidewall provides the benefit that the user may e.g. use a thumb and an index or middle finger to induce a torque or angular momentum onto the medicament container located inside the hollow interior space of the body.

Generally, providing of at least one through opening extending through the sidewall of the body enables to transfer an angular momentum or torque from outside the body into the interior of the body so as to rotate the medicament container located inside the body from outside the body.

According to another example the second longitudinal end of the body comprises a connector operable to mechanically connect with a corresponding counter-connector of another or further housing component of the injection device. Typically, the further housing component of the injection device comprises or conforms a body of the injection device, e.g. configured to receive or to accommodate a drive mechanism to operably engage with the medicament container located inside the presently described elongated housing component.

With some examples the elongated housing component comprises or forms a cartridge holder configured to receive a cartridge forming or constituting the medicament container. The cartridge typically comprises a tubular shaped barrel filled with the liquid medicament. The barrel may be sealed in proximal direction by a bung or stopper displaceably located inside the barrel. The distal end of the barrel may be sealed by a pierceable seal, e.g. a septum. Typically, the first longitudinal end of the body is connectable, e.g. releasably connectable to a piercing assembly, such as a needle assembly or a needle hub.

The second longitudinal end, e.g. the connector of the elongated housing may be configured to non-releasably connect to the further housing component. Then, the elongated housing component is designed and configured as a housing component of a disposable injection device, e.g. configured or intended to be discarded in its entirety when the medicament provided in the medicament container has been used up.

With other examples the elongated housing component is a housing component of a reusable injection device. Here, the connector provided at the second longitudinal end of the elongated housing component is configured to releasably connect to the further housing component of the injection device. In this way and when the content of the medicament container has been dispensed or used up the elongated housing component can be disconnected from the further housing component thus allowing to replace the medicament container.

After or upon inserting of a new, i.e. filled medicament container into the elongated housing component the medicament container may be rotated with its longitudinal axis as an axis of rotation so as to bring a label or some other type of visual information provided on the medicament container in an overlapping configuration with the magnifier of the elongated housing component.

In another aspect the present disclosure relates to an injection device for injecting of a dose of a liquid medicament. The injection device comprises a housing comprising an elongated housing component as described above and another or a further housing component connectable to or being connected to the elongated housing component. The elongated housing component is configured to accommodate a medicament container at least partially filled with the liquid medicament. The further housing component is sized and/or configured to accommodate a drive mechanism. The drive mechanism may be configured or operable to engage with the medicament container to expel or to withdraw a dose of the liquid medicament from the medicament container.

With some examples the drive mechanism comprises a piston rod operable to apply a distally directed pressure onto a bung or piston of the medicament container thereby increasing the inside pressure inside the medicament container in order to expel a dose of the medicament through a distally located outlet.

According to another example the injection device further comprises a medicament container that is filled with the liquid medicament. The medicament container is arranged inside the housing. Typically, the medicament container is located inside the elongated housing component, e.g. inside a cartridge holder. With some examples the medicament container is a cartridge, e.g. comprising a tubular-shaped barrel sealed in proximal longitudinal direction by a piston movably disposed inside the barrel.

The injection devices disclosed herein are not limited to specific embodiments or examples but comprise any combination of elements of different embodiments or examples. Insofar, the present disclosure covers any combination of described embodiments and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful with embodiment of the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of an elongated housing component and an injection device will be described in greater detail by making reference to the drawings, in which:

FIG. 1 schematically illustrates an example of a drug delivery device implemented as an injection device,

FIG. 2 schematically illustrates numerous components of the drug delivery device of FIG. 1,

FIG. 3 is a perspective illustration of a cartridge holder of the injection device,

FIG. 4 is a front view of the cartridge holder of FIG. 3 as seen from the distal end,

FIG. 5 is a front view of the cartridge holder of FIG. 3 as seen from the proximal end,

FIG. 6 is a side view of the cartridge holder of FIG. 3,

FIG. 7 is another side view of the cartridge holder of FIG. 3,

FIG. 8 shows another example of a cartridge holder before insertion of a cartridge,

FIG. 9 shows the cartridge holder of FIG. 8 with the cartridge inserted therein and

FIG. 10 shows a further example of a cartridge holder.

### DETAILED DESCRIPTION

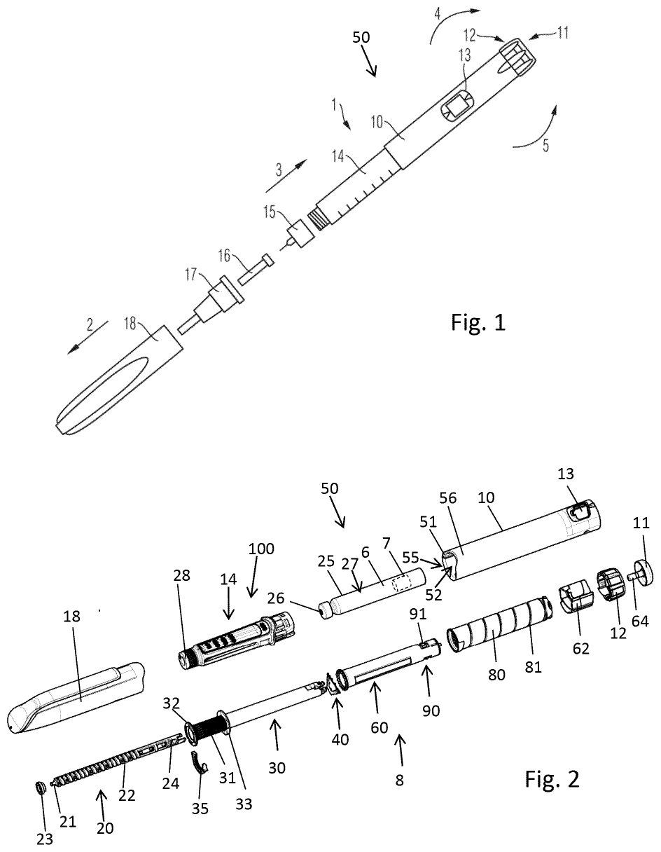

One example of a drug delivery device 1 for administering of a dose of a medicament 27 is illustrated in FIGS. 1 and 2. The drug delivery device 1 is implemented as an injection device. The injection device 1 is a handheld pen-type injector. The injection device 1 may be implemented as a disposable injection device. It may comprise a pre-filled medicament container 6 arranged inside an elongated housing component 100.

The injection device 1 comprises a housing 50. The housing 50 comprises a distal housing component 100 and a proximal housing component 10. As illustrated, both housing components 100, 10 are implemented as elongated housing components extending along a longitudinal direction. When the housing 50 is assembled a proximal end of the distal housing component 100 is connected to a distal end of the proximal housing component 10.

In the presently illustrated example the elongated housing component 100 is a cartridge holder 14 configured to receive or to accommodate the medicament container 6 implemented as a cartridge. With a disposable injection device 1, the cartridge holder 14 may be non-detachably connected to the further housing component 10.

With other examples, the injection device 1 is a re-usable injection device, wherein the cartridge holder 14 is detachably connected to the body 10 for replacing an empty medicament container 6. At or near a distal end of the housing 50, hence at the distal end of the elongated housing component 100 or cartridge holder 14, there is provided a socket 28 configured to mount or to engage with an injection needle 15. The socket 28 may be implemented as a threaded socket and the injection needle 15 may comprise a needle hub being correspondingly threaded to provide a threaded engagement with the socket 28.

Typically, the injection needle 15 is protected by an inner needle cap 16 and either by an outer needle cap 17 and/or a protective cap 18 that is configured to enclose and to protect a distal section of the housing 50 of the injection device 1. Typically, the protective cap 18 is configured to receive the elongated housing component 100 therein. The proximal housing component 10 may comprise and form a body or main housing part configured to accommodate a drive mechanism 8 as shown in FIG. 2. The cartridge holder 14 may be permanently or releasably connected to the body or main housing.

The medicament container 6 comprises a cylindrically-shaped or tubular-shaped barrel 25 sealed in proximal direction 3 by a bung 7 located inside the barrel 25. The medicament container 6 may be prefilled with a liquid medicament 27. The bung 7 is displaceable relative to the barrel 25 of the medicament container 6 in a distal direction 2 through a piston rod 20 of the drive mechanism 8. A distal end of the cartridge 6 is sealed by a pierceable seal 26 configured as a septum and being pierceable by a proximally directed tipped end of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU), wherein 1 IU is the biological equivalent of about 45.5 μg of pure crystalline insulin (1/22 mg). The dose dial 12 may comprise a sleeve shaped knob at the proximal end of the housing 50 of the injection device 1.

As shown further in FIGS. 1 and 2, the housing component 10 comprises a dosage window 13 that may comprise an aperture in the sidewall of the housing component 10. The dosage window 13 permits a user to view a limited portion of a number sleeve 80 that is configured to move when the dose dial 12 is turned. The number sleeve 80 and the dosage window 13 provide a visual indication of a dose currently set. The dose dial 12 may be rotated on a helical path with respect to the housing component 10 when turned during setting and/or dispensing or expelling of a dose.

With some injection devices, the dose dial 12 may be locked in longitudinal direction to the body 10. It may be then limited to a rotational movement relative to the body 10 for setting of the dose.

The injection device 1 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in the dosage window 13 will be ejected from injection device 1.

In the illustrated embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

At least some components of an example of a drive mechanism 8 are illustrated in more detail in FIG. 2. The drive mechanism 8 comprises numerous mechanically interacting components. A flange like support of the housing component 10 comprises a threaded axial through opening threadedly engaged with a thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 50 thereby experiencing a distally directed advancing motion relative to the housing 50 and hence relative to the barrel 25 of the cartridge 6. As a consequence, the bung 7 of the cartridge 6 is displaced in distal direction 2 by a well-defined distance due to the threaded engagement of the piston rod 20 with the housing component 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve or number sleeve 80. The number sleeve 80 is located radially inward of the housing 10. A helical groove 81 is provided about an outer surface of the number sleeve 80. Through the dosage window 13 a part of the outer surface of the number sleeve 80 can be seen. The body 10 is further provided with a helical rib at an inside sidewall portion of an insert piece 62, which helical rib is to be seated in the helical groove 81 of the number sleeve 80. The tubular shaped insert piece 62 is inserted into the proximal end of the further housing component 10, e.g. the body. Alternatively, such a helical rib may be also provided directly on an inside of the sidewall of the housing component 10. The helical rib as well as the insert piece 62 is rotationally and axially fixed to the body 10. There may be provided first and second stops on the housing component 10 to limit a dose setting procedure during which the number sleeve 80 is rotated in a helical motion relative to the housing component 10.

The dose dial 12 in form of a dose dial grip is disposed about an outer surface of the proximal end of the number sleeve 80. An outer diameter of the dose dial 12 typically corresponds to and matches with the outer diameter of a proximal end of the housing component 10. The dose dial 12 is secured to the number sleeve 80 to prevent relative movement there between.

A trigger 11, also denoted as dose button is substantially T-shaped. It is provided at a proximal end of the injection device 10. A stem 64 of the trigger 11 extends into the dose dial 12. The stem 64 and hence the trigger 11 is retained for limited axial movement relative to the number sleeve 80. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 60 engaged, the drive sleeve 30, the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 to prevent further movement in a first direction of rotation, e.g. in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

There may be further provided a ratchet mechanism 90 comprise at least one ratchet feature 91, such as a flexible arm on the sidewall of the tubular-shaped clutch 60. The at least one ratchet feature 91 may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism 90 allows and supports a rotation of the number sleeve 80 relative to the clutch 60 along a second direction of rotation 5, which rotation is accompanied by a regular clicking of the flexible arm of the clutch 60. An angular momentum applied to the number sleeve 80 along the first direction of rotation 4 is unalterably transferred to the clutch 60. Here, the mutually corresponding ratchet features of the ratchet mechanism 90 provide a torque transmission from the number sleeve 80 to the clutch 60.

The number sleeve 80, the dose dial 12 and the trigger may form part of a dial extension, hence and assembly of components of the drive mechanism 8 that starts to extend or to displace from the proximal end of the body 10 as a dose is dialed. During dispensing of a dose, hence when a user depresses the trigger 11 in distal direction 2, the dial extension is subject to a distally directed movement relative to the body 10, hence along the distal direction 2. During such a dispensing motion, the number sleeve 80 is subject to a rotation along a dose decrementing direction 5, e.g. counter-clockwise. The drive mechanism as described above is explained in more detail e.g. in WO2004/078239A1, WO 2004/078240A1 or WO 2004/078241A1 the entirety of which being incorporated herein by reference.

The expelling mechanism or drive mechanism 8 as described above is only exemplary for one of a plurality of differently configured drive mechanisms that are generally implementable in a disposable or re-usable pen-injector.

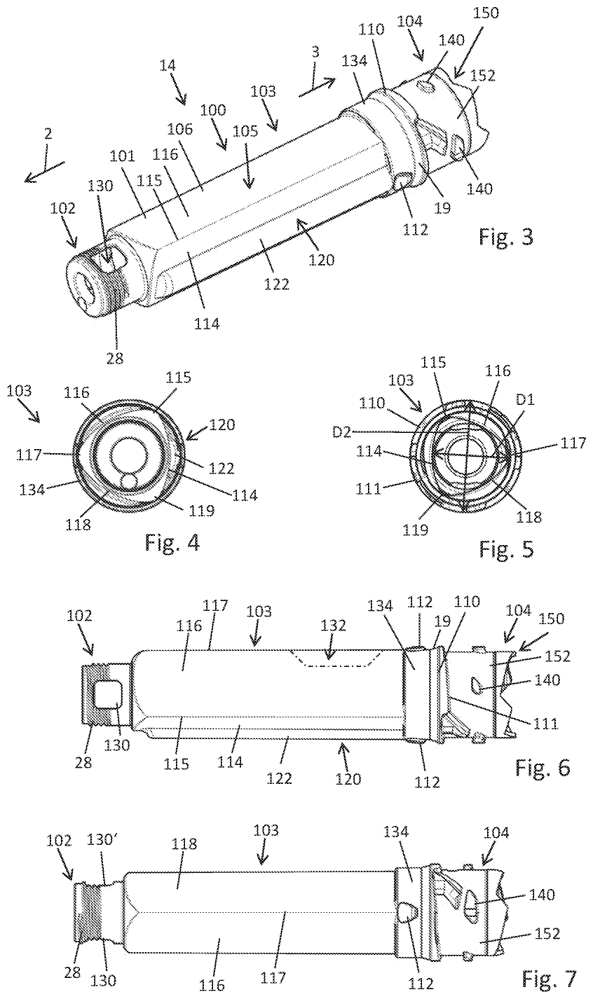

In FIGS. 3-7 an example of the elongated housing component 100 is illustrated in greater detail. The elongated housing component 100, e.g. the cartridge holder 14, comprises an elongated body 101. The body 101 comprises a first longitudinal end 102, an intermediate section 103 and a second longitudinal end 104.

The first longitudinal end 102 is a distal end of the cartridge holder 14 and the second longitudinal end 104 is a proximal end of the cartridge holder 14. The body 101 comprises a sidewall 106 extending from the first longitudinal end 102 to the second longitudinal end 104. The sidewall 106 forms or confines a hollow interior space 105. The hollow interior space 105 is sized to accommodate the medicament container 6.

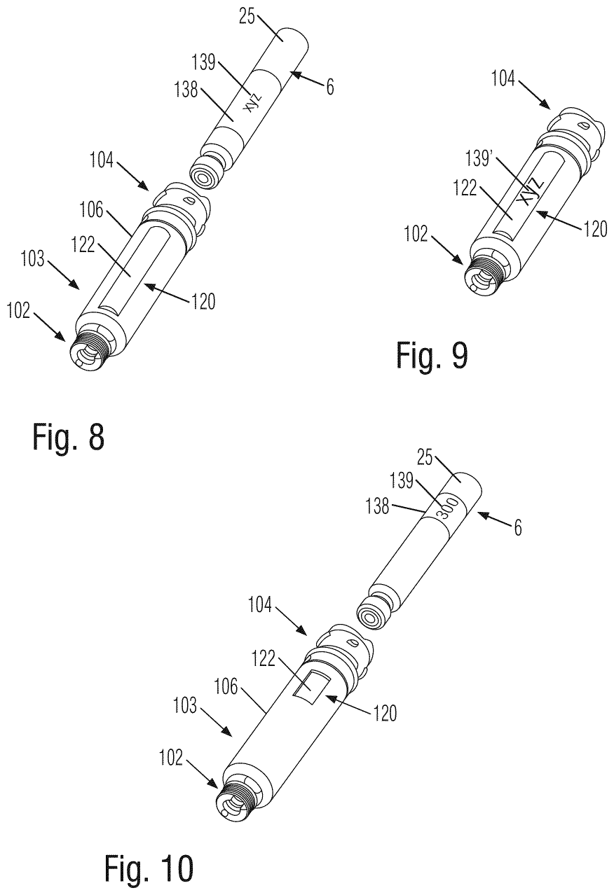

As illustrated in FIGS. 3-7 the body 101 comprises a magnifier 120. The magnifier 120 is integrally formed with the sidewall 106. With other examples the magnifier 120 extends through a respective aperture of the sidewall 106. The magnifier 120 allows and/or enables visual inspection of the hollow interior space 105 from outside the body 101. The magnifier 120 comprises a bulged section 122. The bulged section 122, hence the magnifier 120 forms a magnifying optical lens. The bulged section 122 bulges radially outwardly from the sidewall 106 of the body 101. In this way and since the magnifier 120 is made of a translucent or transparent material, e.g. a plastic material, a label 138 of the medicament container 6, e.g. provided with visual information 139 as illustrated in FIG. 8 becomes visible from outside the body 101 when located inside the hollow interior 105.

If the label 138 and hence the visual information 139 overlaps with the magnifier 120 the visual appearance 139′ of the visual information 139 and hence of the label 138 is magnified and enlarged as becomes apparent from a comparison of FIGS. 8 and 9.

Contrary to the embodiment as shown in FIGS. 3-7, the example of the elongated housing component 100 as illustrated in FIGS. 8-10 comprises a body 101 with an intermediate section 103 of tubular shape. With the examples of FIG. 3 and FIG. 8 the magnifier 120, in particular its radially outwardly extending bulged section 122, extends all along the intermediate section 103. Here, the magnifier 120 is of elongated shape and extends parallel to a longitudinal axis of the body 101.

With the further example as illustrated in FIG. 10 the intermediate section 103 is also of tubular shape. But there, the magnifier 122 is located near or adjacent to the second longitudinal end 104 of the body 101. The longitudinal extent of the magnifier 120 of FIG. 10 is shorter compared to the magnifier 120 of the example of FIG. 3 or 8. With the example of FIG. 10, only a selected portion, e.g. a proximal end section of the medicament container 6 will be magnified. Here, the barrel 25 of the respective medicament container 6 is provided with visible information 139 at a particular longitudinal portion, e.g. at a predefined longitudinal position of the elongated or tubular shaped medicament container 6.

When the medicament container 6 has reached its final position inside the hollow interior 105 of the elongated housing component 100 the label 138 and/or its visual information 139 longitudinal overlaps with the position or longitudinal extent of the magnifier 120.

As illustrated in FIGS. 3-10 the body 101 comprises at least one through opening 130 extending through the sidewall 106. There, the through opening 130 is provided in the stepped down threaded socket 28 of the body 101. The through opening 130 is sized to receive at least a fingertip of a user of the injection device 1. In this way the at least one through opening 130 provides access to the interior space 105. With the medicament container 6 assembled inside the body 101 the user gains access at least to an outside surface of the sidewall of the medicament container 6 and is enabled to induce an angular momentum or torque onto the medicament container 6 from outside the body 101 relative to the body 101.

In this way the user is given a possibility to modify the angular position or angular orientation of the medicament container 6 inside the hollow interior space 105 even when the proximal end 104 of the elongated housing component 100 is attached to the proximal housing component 10. By rotating the medicament container 6 relative to the body 101 the label 138 and/or of the visual information 139 can assume a radial overlapping configuration with the magnifier 120.

As it becomes apparent from the side view of FIG. 7 the sidewall 106 of the body 101 comprises a first through opening 130 and a second through opening 130′. The first and the second through openings 130, 130′ are located at radially and hence diametrically opposite positions. This allows to provide access to the interior space 105 from opposite radial positions. In this way, a user may use a thumb and at least one of an index or middle finger to induce an angular momentum or torque onto the medicament container 6 when assembled inside the body 101.

As further indicated in FIG. 6 the through opening 132 may be provided elsewhere, e.g. at an arbitrary longitudinal position of the sidewall 106, e.g. at or in the intermediate section 103.

In the example as illustrated in FIGS. 3-7 the elongated housing component 100 is intended for releasable connection with the proximal housing component 10. Here, the second longitudinal end 104 comprises a connector 150 operable or configured to mechanically connected with a corresponding counter-connector 55 of the proximal housing component 10 of the injection device 1 as illustrated in FIG. 2. At least one of the connector 150 and the counter connector 55 comprises an insert section 152 configured to be received in a complementary shaped receptacle 52 of the other one of the connector 150 and the counter connector 55. Hence, one of the elongated housing component 100 and the proximal housing component 10 comprises an insert section 152 shaped and configured for longitudinal insertion into a complementary shaped receptacle of the other one of the elongated housing component 100 and the proximal housing component 10.

As illustrated, the counter-connector 55 of the proximal housing component 10 comprises the receptacle 52 which is configured to receive the insert section 152 forming the proximal end 104 of the elongated housing component 104. On an outside surface of the insert section 152 there are provided numerous fastening features 140, e.g. implemented as a radial protrusion protruding radially outwardly from a sidewall of the insert section 152. On an inside of the receptacle 52 of a sidewall 56 of the proximal housing component 10 there are provided correspondingly shaped recesses, e.g. to form a snap fit or to threadedly engage with the fastening features 140. The insert section 152 and the receptacle 52 may engage by way of a bayonet joint.

An insert motion of the insert section 152 into the receptacle 52 is delimited by a radially outwardly protruding flange 110 of the body 101. The flange 110 protrudes radially outward the at a distal end of the insert section 152. The flange 110 confines the longitudinal extent of the insert section 152. The flange 110 comprises a proximally facing abutment face 111 to longitudinally abut with a correspondingly shaped distally facing abutment face 51 of the sidewall 56 of the proximal housing component 10.

The flange 110 further comprises a distally facing abutment face 19. The distally facing abutment face 19 may be configured to abut with a proximal end face of the protective cap 18 when assembled onto the housing 50, thereby covering at least a portion of the elongated housing component 100 or of the cartridge holder 14.

Axially or longitudinally adjacent to the flange 110 the body 101 comprises an annular rim 134 of tubular shape. The annular rim 134 may provide a form fitting or friction fitting engagement with an inside surface of the proximal end of the protective cap 18. On the rim 134 there is provided at least one protrusion 112. The protrusion 112 may form a snap fit engagement, e.g. with a correspondingly shaped recessed structure on the inside surface of the protective cap 18.

As illustrated in FIG. 6 there may be provided diametrically oppositely located protrusions 112 by way of which the mechanical engagement between the distal housing component 100 and the protective cap 18 can be improved.

In the example as illustrated in FIG. 3 the intermediate section 103 of the sidewall 106 of the body 101 is of polygonal shape. The intermediate section 103 comprises a first sidewall section 114, a second sidewall section 116 and a third side wall section 118. As seen in a circumferential or tangential direction the first sidewall section 114 adjoins the second side wall section 116 via or across a first ridge portion 115. The second side wall section adjoins the third sidewall section 118 along the respective tangential direction across or via a second ridge portion 117. Finally, the third sidewall section adjoins in tangential direction the first sidewall section 114 along, across or via a third ridge portion 119.

The crest of the ridge portions 115, 117, 119 and the respective outside surfaces of the crests of the respective ridge portions 115, 117, 119 may coincide with the outer radial position of the rim 134. Hence, as seen in longitudinal direction, the radial outer side of the ridge portions 115, 117, 119 may align with the outside surface of the rim 134. In this way and when inserting the cartridge holder 14 into the cup-shaped receptacle of the protective cap 18, an inside surface of the protective cap 18 may slide on the ridge portions 115, 117, 119 until the proximal portion of the protective cap 18 smoothly engages with the rim 134.

The sidewall sections 114, 116, 118 located circumferentially between the ridge portions 115, 117, 119 are somewhat radially recessed from the imaginary circumference of the rim 138. In this way, the radially outwardly bulged section 122 is allowed to protrude radially outwardly from the first sidewall section 114 as illustrated in FIGS. 3 and 4. An outside surface of the radially outwardly bulged section 122 is still located radially inside the imaginary tubular shaped longitudinal extension of the rim 134. In this way, the radially outwardly bulged section 122 of the magnifier 120 does not interfere with an assembly of the protective cap 18 onto the distal housing component 100.

As further illustrated in FIG. 5 the intermediate section 103 comprises a first outer diameter D1, a respective first outer circumference or cross-section. The second longitudinal end 104 or the rim 134 comprises a second outer diameter D2 or a respective second outer cross-sectional circumference. The first outer diameter D1 is smaller than the second outer diameter D2. In this way there is provided sufficient space in the intermediate section for a radially outwardly bulged section of a magnifier 120.

### LIST OF REFERENCE NUMBERS

1 injection device
2 distal direction
3 proximal direction
4 dose incrementing direction
5 dose decrementing direction
6 medicament container
7 bung
8 drive mechanism
10 housing
11 trigger
12 dose dial
13 dosage window
14 cartridge holder
15 injection needle
16 inner needle cap
17 outer needle cap
18 protective cap
19 abutment face
20 piston rod
21 bearing
22 first thread
23 pressure foot
24 second thread
25 barrel
26 seal
27 medicament
28 threaded socket
30 drive sleeve
31 threaded section
32 flange
33 flange
35 last dose limiter
40 spring
50 housing
51 abutment face
52 receptacle
55 counter connector
56 sidewall
60 clutch
62 insert piece
64 stem
80 number sleeve
81 groove
90 ratchet mechanism
91 ratchet feature
100 housing component
101 body
102 longitudinal end
103 intermediate section
104 longitudinal end
105 interior space
106 sidewall
110 flange
111 abutment face
112 protrusion
114 sidewall section
115 ridge portion
116 sidewall section
117 ridge portion
118 sidewall section
119 ridge portion
120 magnifier
122 bulged section
130 through opening
132 through opening
134 rim
138 label
139 visual information
140 fastening feature
150 connector
152 insert section

1.-15. (canceled)

16. An elongated housing component of an injection device, the elongated housing component comprising:
a body comprising a first longitudinal end, an intermediate section, and a second longitudinal end opposite to the first longitudinal end, wherein the intermediate section is located longitudinally between the first longitudinal end and the second longitudinal end,
wherein the body comprises a sidewall extending from the first longitudinal end to the second longitudinal end, the sidewall confining a hollow interior space sized to accommodate a medicament container, and
a magnifier extending through the sidewall or integrated into the sidewall to increase a visual appearance of at least a portion of the medicament container when the medicament container is inside the hollow interior space.

17. The elongated housing component of claim 16, wherein the magnifier comprises a bulged section extending outwardly from the sidewall.

18. The elongated housing component of claim 16, wherein the magnifier is elongated and extends in a direction parallel to a longitudinal axis of the body.

19. The elongated housing component of claim 16, wherein the magnifier is located in the intermediate section of the body.

20. The elongated housing component of claim 16, wherein the magnifier extends along the intermediate section of the body.

21. The elongated housing component of claim 16, wherein the intermediate section comprises a first outer diameter, the second longitudinal end comprises a second outer diameter, and the first outer diameter is smaller than the second outer diameter.

22. The elongated housing component of claim 16, wherein:
the intermediate section comprises a polygonal cross-section with at least a first sidewall section, a second sidewall section, and a third sidewall section, and
the first sidewall section, the second sidewall section, and the third sidewall section are adjoined to each other along a first or second tangential direction.

23. The elongated housing component of claim 22, wherein:
the first sidewall section adjoins in the first tangential direction to the second sidewall section along a first ridge portion,
the second sidewall section adjoins in the first tangential direction to the third sidewall section along a second ridge portion,
the first sidewall section adjoins in the second tangential direction to the third sidewall section along a third ridge portion, and
the second tangential direction is opposite to the first tangential direction.

24. The elongated housing component of claim 23, wherein the magnifier is located on the first sidewall section between the first ridge portion and the third ridge portion.

25. The elongated housing component of claim 22, wherein at least the first sidewall section has a convex shape along the first tangential direction.

26. The elongated housing component of claim 16, wherein the body comprises at least one through opening extending through the sidewall to provide access to the medicament container when the medicament container is in the body.

27. The elongated housing component of claim 26, wherein the at least one through opening comprises a first through opening and a second through opening, the first through opening and the second through opening extending through the sidewall, and the first through opening is located diametrically opposite to the second through opening.

28. The elongated housing component of claim 16, wherein the second longitudinal end comprises a connector operable to mechanically connect to a corresponding connector of a further housing component of the injection device.

29. The elongated housing component of claim 16, wherein the magnifier (i) comprises a bulged section extending outwardly from the sidewall, (ii) is elongated and extends in a direction parallel to a longitudinal axis of the body, (iii) is located in the intermediate section of the body, and (iv) extends along the intermediate section of the body.

30. The elongated housing component of claim 29, wherein the body comprises a first through opening and a second through opening, the first through opening and the second through opening extending through the sidewall to provide access to the medicament container when the medicament container is in the body, and the first through opening being located diametrically opposite to the second through opening.

31. The elongated housing component of claim 29, wherein:
the intermediate section comprises a polygonal cross-section with at least a first sidewall section, a second sidewall section, and a third sidewall section,
the first sidewall section adjoins in a first tangential direction to the second sidewall section along a first ridge portion,
the second sidewall section adjoins in the first tangential direction to the third sidewall section along a second ridge portion,
the first sidewall section adjoins in a second tangential direction to the third sidewall section along a third ridge portion,
the second tangential direction is opposite to the first tangential direction,
the magnifier is located on the first sidewall section between the first ridge portion and the third ridge portion, and
at least the first sidewall section has a convex shape along the first tangential direction.

32. An injection device for injecting of a dose of a liquid medicament, the injection device comprising:
a first housing comprising an elongated housing component, the elongated housing component being configured to accommodate a medicament container at least partially filled with the liquid medicament;
a magnifier extending through a sidewall of the elongated housing component or integrated into the sidewall to increase a visual appearance of at least a portion of the medicament container when the medicament container is arranged in the elongated housing component; and
a second housing component connectable or connected to the elongated housing component, the second housing component being sized or configured to accommodate a drive mechanism configured to engage with the medicament container to expel or to withdraw the dose of the liquid medicament from the medicament container.

33. The injection device of claim 32, further comprising the medicament container arranged inside the elongated housing component, the medicament container at least partially filled with the liquid medicament.

34. The injection device of claim 32, wherein the magnifier comprises a bulged section extending outwardly from the sidewall of the elongated housing component.

35. The injection device of claim 34, wherein the magnifier is configured to magnify at least the portion of the medicament container.
